(19) United States
(12) Patent Application Publication
(10) Pub. No.: US 2018/0271176 A1
(43) Pub. Date: Sep. 27, 2018

(54) UNDERGARMENTS WITH MULTI-LAYER BARRIERS TO LESSEN EMF/EMR/EHS EMITTING FEATURES AND MOISTURE MANAGEMENT PROPERTIES

(71) Applicant: Delores Harrell, Ellenwood, GA (US)

(72) Inventor: Delores Harrell, Ellenwood, GA (US)

(21) Appl. No.: 15/912,905

(22) Filed: Mar. 6, 2018

Related U.S. Application Data
(60) Provisional application No. 62/469,478, filed on Mar. 9, 2017.

(51) Int. Cl. A41B 9/12; A41B 9/06; A41B 9/02; A41B 13/08; A41B 9/04; A41C 3/00; A41B 17/00; A41B 13/04; A41C 3/12; B32B 5/02; B32B 5/26; A61F 13/84

(52) U.S. Cl. CPC A41B 9/12; A61F 2013/8408; A41B 9/02; A41B 13/08; A41B 9/04; A41C 3/005; A41B 17/00; A41B 13/04; A41C 3/12; B32B 5/02; B32B 5/26; A61F 13/8405; A41B 2400/52; A41B 2400/62; B32B 2307/212; B32B 2555/02; A41B 9/06

(57) ABSTRACT

Undergarments are worn beneath other clothing articles and have direct contact to human skin and vital body organs. The rationale behind the development of this invention is to minimize electromagnetic fields/frequency (EMF); electromagnetic radiation (EMR); and electromagnetic hypersensitivity (EHS) while managing moisture production within the same. Multiple textile barrier linings will serve as blocking measures to minimize the hazardous effects linked to various EMR/EMF emitting exposure as well as introduce moisture and absorption properties. In addition, this invention will address the problems with existing products and EMF/EMR protectors that are described as heavy, uncomfortable, and expensive to process. Furthermore, this invention will serve a three-tier purpose in aiding the consumer in the first layer of protection for their genital and upper body torso, while providing a barrier from radiated EMF/EMR energy, as well as offer a composition to eliminate the production of moisture and bacteria through additional blocking features.

### FIELD OF THE INVENTION

The present invention will claim the benefit of the provisional application (No. 62469478 filed on Mar. 9, 2017) and in general relates to undergarments for the upper and lower torso, serving as barriers of protection from electromagnetic radiation and frequency, while also creating another layer of moisture control and bacteria blockage. More particularly, this invention relates to undergarment apparel made of smart-textile composite fabrics that are considered safe for the human skin along with a moisture bacteria absorption barrier.

### BACKGROUND OF THE INVENTION

The prevalence of Electromagnetic Fields and Electromagnetic Radiation (EMF/EMR) and those susceptible to exposure and a reaction to radiation levels, such as Electromagnetic Hyper Sensitive (EHS) have increased substantially in the past decade. Advances in technology and the promotion of electromagnetic pulsating currents are rampant. Prolonged and/or overexposure to electromagnetic fields and radiation waves can contribute to health conditions such as carcinogens or tumor growth, loss of fertility, miscarriage, low sperm production, and other reproductive issues as well as central nerve damage, and endocrine changes. Both genders are susceptible to increased risks of cardiovascular disease, breast cancer and neurodegenerative disorders. EMF/EMR can lower the intracellular defense activity contributing to the development of DNA damage, as well as lead to cognitive difficulties, depression, and anxiety disorders. In young children, the results could lead to slowed motor skills and learning difficulties, autism. and attention issues. Infants exposed to radiofrequency pulsating wave are vulnerable with their underdeveloped skin, skull, and immune system.

Prior art that was developed as a protective shield was often very uncomfortable such as the faraday cage used by safety sensitive personnel for protection against electric shock weapons, and electrical fields. In the U.S. patent publication no. 2011/0258762 composite material was considered heavy and difficult to weave and ultimately prohibited the fabric to be open and with adequate ventilation. Similarly, the U.S. patent 20140220845 was developed for heavy ballistic, and spike or stab threats, though it provided some multilayer elements and fabrics with breathable elements, its overall functions were too abrasive for any intimate apparel or undergarment wear. Other patents like U.S. 20140246609; U.S. 20160149293; and U.S. 20160149293 are body shields for portable devices and have been found to be unsafe for direct human skin contact. Another patent WO1996040362 required electrical conduction and is still considered heavy material that was unflattering and unsafe for direct skin contact.

In the (WO2016092399) patent, the overall intent was good however, water is a compound that is difficult to move and counterproductive for consumers that are trying to steer away from heavy moisture in fabric. Studies have found that too much moisture leads to high rates of bacteria, odor, and blocks the fabric's proper ventilation. In addition, the attenuation ranges and specific fabrics discussed are not conducive for intimate and/or undergarment apparel. Lastly, some textile materials can result in more harm to the consumer as well as the ecological system based on their composition.

Additional, prior art that attempted to meet the integral needs of consumers have relied on composite materials that have lead to consumer complaints based on the quality and consistency of the fabric. Furthermore, several mechanisms in the prior applications have shown failure rates, created health-related symptoms for the consumer, poised environmental and safety risks, as well as manufacturing defects and structural integrity concerns. For example, the U.S. Pat. No. 5,932,496 contained thick composite fibers of cloth that have resin permeating between individual fibers along with embedded identification devices and chips, making this invention unparalleled to the embodiment of the invention described herein. Furthermore, patent CN 103129035 was developed as an anti-radiation fabric with layers and a waterproof breathable layer, that provided some genuine intentions. However, the CN 103129035 composite material contained toxic hazards and health risks from applications of Nanosilver, which has been found to be an environmental hazard. These risks to the human body include problems in the lung, gastrointestinal track, and skin discoloration. Further health-related complications have been found as a result of Nanosilver in affecting two major body organs the liver and spleen.

Two patents by Gray, U.S. Pat. No. 6,488,564 and U.S. Pat. No. 6,665,877 had helpful ambitions as to creating air ions to conceal electrostatic charges at the source on the genital or breast cavity as well as in the near vicinity. This inventor further utilized animal studies to support that electrostatic fields have an influence on cells inside living tissues that might contribute to cancer growth. However, the main drawback in these inventions was the primary focus on the interaction and/or static charges created as a result of direct clothing friction, leading to generated electrostatic fields. Another drawback with these two former art(s) was that technology and production of composite matters have drastically changed in the past decade. Furthermore, these studies do not specifically address the imminent risk from electromagnetic radiation and radiofrequency pulsating fields. These prior applications focused on the probable conductivity from body to surface areas and failed to include the environmental factors, and the growth of electromagnetic and radiofrequency radicals from physical and intermittent oscillating properties. Another promising invention by Gray was WO 96/40362; the goal of this invention was to extrapolate sweat and moisture away from the body, while blocking EMF emitting radicals from the genital and breast cavity area. In this invention, the consumer is subject to a field attenuating article that is created by friction of the clothing being in close proximity to attenuate an electrostatic field. In sum, the patent by Gray WO 96/40362 adopts the same issues in failing to included the environmental risk factors as well as the technological advances as a result of the development of pulsating radiofrequency and electromagnetic properties.

Another invention, U.S. Pat. No. 9,210,956B2 referenced the “efficacious” properties of the brassiere, without denoting the potential health consequences and residual effects. In the claims for this invention, it was reported that the silver mesh fabric would serve as the EMF shield and that the attenuating effects could gauge up to 18 GHz The inventor further mitigates the problem of the invention by offering plural protective layers that are likely to result in prolonged skin exposure, discoloration, and risk to the breast cavity and nearby organs. This invention primarily focused on Eddy currents however there are other types of EMF that might be statically or dynamically induced. In addition, other radiating properties can be emitted through Back EMF and magnetic damping.

The reliance of wireless and cellular devices has grown to insurmountable numbers over the past two decades. Millions of consumers rely on wireless and cellular devices for pleasure, work, and school, creating further health and environmental concerns due to the continued and prolonged exposure to EMF/EMR. There are reported biological effects from radiofrequency waves from cell towers, Wi-Fi, wireless laptops, smart meters, cell phones, bluetooths, baby monitors, and cordless phones. It has also been reported that the environmental and toxicological elements are incessant when consumers are in congested areas like airports, sports arenas, and malls. Task force groups, scientist, medical investigators, epidemiologist, academia's, health advocates are all raising awareness of the potential harmful effects from EN/IF/EMIR exposure as technology continues to advance. Prior art was not aware of the potential technology growth or public health and environmental risk that would result. In addition, the exposure and risk for infants, children, and vulnerable populations was not foreseen. The wireless and cellular devices have been adopted into the daily lifestyle of consumers including their leisure or recreational outings, as well as home and work environments. Furthermore, the pivotal growth in epidemiological and independent studies continue to raise further concern over the potential risk factors associated with pulsating radicals.

The public health risk are rising and consumers need primary preventive measures, which will be obtained through this patent application. The latency period for some cancerous tumors could be 15 to 30 years, epidemiological studies are ongoing and are suggestive that prolonged and frequent exposure to radiofrequency can lead to more harm for the fetus and young children. Additionally, some of the current research and safety guidelines that are being endorsed are outdated based on current technology use of 4G, 4G lte, and the arising 5G. The safety regulations for cell phone use is currently inconsistent with the practical human consumer use, since the applications were conducted with plastic mannequins that lacked any consistency with emitting pulsating effects similar to humans, making those guidelines implausible. Consumers need products that will address these issues and minimize their overall daily exposure. In addition, the advancements in 5 G have promised greater intensity and faster speed that will increase the emitting radiation and pulsation wave exposure to all consumers and those that are in close proximity.

Based on these limitations, the novel invention was developed to overcome the aforementioned obstacles. The present invention seeks to offer a composite material having improved electromagnetic, frequency, and radiation resistant elements, as well as provide moisture absorption tolerance. Humans are at risk of EN/IF/EMIR exposure based on their occupational work, travel through airports, scanning machines, venues with high traffic like sports arenas and malls, medical x-rays, microwaves, Wi-Fi and cell towers, smart meters, as well as high voltage power lines. The development of undergarments/intimate apparel with embedded conductive inner linings will reduce exposure to electromagnetic and radiofrequency pulsating fields of different strengths, while further acting as a guard to block moisture and bacteria producing elements.

### SUMMARY OF THE INVENTION

This novel invention will be inspired by high engineered smart-textile composite fabrics with a superior success rate of applying barrier applications, from environmental EMF/EMR factors and moisture producing elements. It is further intended that this invention will address forces of static and magnetic substance. Advances in technology contribute to the electromagnetic forces being on a continuous move which contributes to other electromagnetic frequencies and radiation actions. Consumers are becoming more proactive about their health and are seeking ways to minimize their risk factors. EMF/EMR are a constant health hazard but also serve a functional role in the technological advances that they provide. Targeted populations will include individuals with high levels of susceptibility to electromagnetic (EHS), and radiofrequency radiation, cancer surviving victims and high-risk subjects, as well as health conscientious consumers. Individuals that are at occupational or residential risk include those that are working or living in environments with high frequency voltage such as airports, court systems, medical facilities, smart meters, cellular and Wi-Fi towers. as well as daily usage of cell phones and other technologies.

The embodiments of this invention will include hybrid knitted yarns, and/or bamboo along with 30% silver EMF/EMR blocking attenuation effects, that are in the low frequency range and still meet the standards of shielding the consumer. In addition, all products will be safe on the human skin, eco-friendly, provide antibacterial and absorption blocking properties, as well as reduce microbial growth. The purpose of this invention is to develop undergarment(s) apparel with a conductive sheath of protecting EMF/EMR layer to lessen exposure to radiation for the reproductive organs, pelvic and breast cavity area, as well as offer a buffer of moisture absorption fabric. Intimate apparel will be designed to block moisture production, shield electromagnetic radioactive emitting exposure, and reduce the risk of damage to cells in the reproductive organs that will include genital, groin, pelvic, crotch, and breast cavity areas. The primary goal in this novel invention is to limit EMF/EMR exposure and electromagnetic radiation and reduce these risks through eco-friendly intimate apparel with moisture blocking properties. The conductive barrier will significantly reduce the amount of exposure to electromagnetic, pulsating, and radio-frequency hazards that have been linked to several health risks.

The following terms should be considered inter-changeable in that they all serve in functional roles of undergarments, including underwear, and intimate apparel. Undergarments are coverings that protect and serve as the initial covering for the intricate sexual and reproductive organs in the human body, i.e. the genital, pelvic, and breast anatomy. All intimate undergarment apparel will have an embedded shield to reduce the surface area from exposure, that serves as a barrier or shield during prolonged/over exposure, while also having a moisture protection blockage. The undergarment/intimate apparel will include any of the following types that are the initial coverings on the human body, to include bras, bralettes, brassieres, sports shapewear, strapless, sports bras, pull over/slip on, nursing, and breast shielding pads; panties, low-rise hipsters, high-cut briefs, classic briefs, bikinis, boy shorts, tanga, and female boxers. Additional undergarment/intimate apparel may include corsets, camisoles, tanks, tube tops, t-shirts, undershirts, body slips, and bodysuits and long underwear. Male underwear will include any of the following: boxers, briefs, boxer briefs, trunks, jockstrap, bikinis, tank tops, t-shirts, undershirts, and long underwear. Further protective under coverings will be developed using the same fabrics and multi-layers to assist infants and individuals that have incontinence needs with sanitary absorption to include cloth diapers, onesie undergarments, and toddler tanks and pull-ups, as well as gender specific briefs. Unisex undergarments will consist of thermal undershirts, t-shirts, underpants, and regular undershirt apparel. These undergarment and intimate apparel will be worn under the primary clothing in direct contact to the physical human anatomy and some vital organs.

Undergarments/intimate apparel will be tailored for females and males with designs for young, middle, and older age spans. Designs will include fashionable lace, satin, spandex, lycra, nylon, organic cotton, non-toxic cotton, polymide, mesh, and features to compliment the product and add cosmetic appeal. The conductive fabric/material will act as an embedded barrier to reduce exposure to electromagnetic pulsating radiation and frequency for consumers that are at risk for damage to cells in the reproductive and breast cavity area. The conductive fabric/material will be interwoven into the undergarment/intimate apparel serving as a primary barrier and the moisture blocking fabric will be in closest proximity to the organs of the skin. Textile production and manufacturing have continued to evolve and many of the specific embodiments in this invention may have digital printing capability with contour seaming, seamless knitting, power paneling, and 3D warp-knitted textiles. The objective of this novel invention is to utilize engineering smart-textile composite fabric designs to develop sophisticated undergarments and apparel, that meets the needs of the modern day and health conscientious consumer.

Keeping with the current trends and cutting edge products, this invention will utilize Swiss Shield® EMF/EMR fabric designs that are custom to having light weight, ecological unbleached, euro-white cotton and yarn fabric. The Swiss Shield® EMF/EMR comes in different fabrications, both the Naturell and Wear productions have the mechanical composition that are appropriate for undergarment apparel. Swiss Shield® yarn with hybrid yarn has shown better shielding in lower to higher frequency ranges and is a good shielding fabric from indoor and outdoor environments. These manufacturers have Oeko-Tex certifications for no harmful substances and other independent testing for shielding performance.

Due to varied technical applications this intervention may be embodied in other formats or specific forms that should not reflect a departure from the primary characteristics accordingly. The Swiss Shield® will serve as the primary fabric based on its universal properties, endorsements, and certificates of Oeko-Tex 100 (Class 1) making it a safe product for baby underwear. Soft & Safe™ will also be applied to undergarment products that are more conducive to higher attenuation ranges and promote additional safety features. The fabric may also include blending of fabrics together to reflect a more conclusive and uniform appeal as well as wear of continuity. In addition, any refinements or upgrades to these two composite barrier wearing fabrics (i.e. Swiss Shield® and Soft & Safe™) will be considered adaptable within the embodiments of this invention. Furthermore, these composites may continue to be subject to advancements in manufacturing and engineering and that will be considered as consistent within the scope of this invention. Other shielding smart-textile fabrics that meet or exceed these specifications may also be adopted into the specification for this patent.

This invention will contain inner porous and absorption properties to channel the moisture to the outer surface, helping to reduce microbial growth. Micromodal and/or mesh fabric with Polygiene® controlling elements as well as moisture blocking properties are ideal for intimate apparel based on the material softness. In Polygiene® applications, the permanent treatment is applied at the finishing stages of textile production and field/lab tests show a high level of odor control beyond the usual lifespan of a garment. This lifespan odor control is possible as the technology is bound and permanently integrated in the fabric and will not wash out. In addition, Polygiene® does not contain the nanosilver elements that have been used in prior inventions. Other antimicrobial applications that meet or exceed these standards may also be adopted into the specifications for this patent.

Thus, the immediate embodiments should therefore be considered as informatory and not regulative of the scope of the invention indicated by the aforementioned claims. All the same, the foregoing description and all changes which come within the significance and compass of comparability of the claims are intended to be entwined in that respect. This invention may involve a consortium with any of the following elements to further the consistency and quality of the property: cotton, nylon, hybrid yarn, silver shielding, and bamboo fibers that are similar in nature and will not be considered mutually exclusive to the merits of this invention. In addition, this invention may include hook and loop fasteners, eco-friendly taping adhesive, clasp, clamps, elastic encasings, and threading with shielding properties. The invention may further consist of cotton, spandex, nylon, polyester, organic blends, natural fibers rayon protection, cotton dyes or other dyes that are found to be non-toxic or hazardous, elastic binding, nickel-free hooks and tools for the body, underarm pits, crotch, lining, trims and side panels.

### BRIEF DESCRIPTION OF THE DRAWINGS

Hereunder is illustrated, in the form of non-limiting examples for the present invention with the aid of the accompanying drawings that are not necessarily drawn to scale, the credence instead is placed upon clearly illustrating the principles of like parts in the various views. In the drawings, like reference numerals designate corresponding parts throughout the various views, and all the views are schematic in which:

FIG. 1 is a view of the articles of fabric composition types that contains EMF/EMR/EHS radiation emitting barrier material along with blocking moisture properties intertwined between two or more articles of original fabric;

FIG. 2 is a dimensional view of the fabric composition types in order that contains EMF/EMR/EHS radiation emitting barrier material coupled with blocking moisture properties intertwined between two or more articles of original fabric;

FIG. 3 is a description of the Swiss Shield® Naturell Shielding composition and performance properties that may be included in the embodiment of this invention;

FIG. 4 is a description of the Swiss Shield® Wear Shielding composition and performance properties that may be included in the embodiment of this invention;

FIG. 5 is a description of the Swiss Shield® Shielding composition that shows the interlocking yarn fibers that may be included in the embodiment of this invention;

FIG. 6 is a description of the Soft & Safe™ Shielding composition and performance properties that may be included in the embodiment of this invention;

FIG. 7 is a description of the Microfiber blocking elements and performance properties that may be included in the embodiment of this invention;

FIG. 8 is a description of the Polygiene® blocking elements and performance properties that may be included in the embodiment of this invention;

FIGS. 9 (A, B, & C) is a view of a (female) tank undergarment that will contain EMF/EMR/EHS barrier materials along with moisture blocking properties;

FIGS. 10 (A & B) is a view of a (male) tank undergarment that will contain EMF/EMR/EHS barrier materials along with moisture blocking properties;

FIGS. 11 (A & B) is a view of a (unisex) shirt undergarment that will contain EMF/EMR/EHS barrier materials along with moisture blocking properties;

FIGS. 12 (A & B) is a view of a (female) panty undergarment that will contain EMF/EMR/EHS barrier materials along with moisture blocking properties;

FIGS. 13(A & B) is a view of a (male) brief undergarment that will contain EMF/EMR/EHS barrier materials along with moisture blocking properties;

FIG. 14 is a view of a (female) under bridge and siding panel base for a bra forming undergarment that will contain EMF/EMR/EHS barrier materials along with moisture blocking properties;

FIG. 15 is a view of a (female) breast cupping, molded, contour appearance for a bra forming undergarment that will contain EMF/EMR/EHS barrier materials along with moisture blocking properties;

FIG. 16 is a view of a (female) nipple shield undergarment that will contain EMF/EMR/EHS barrier materials along with moisture blocking properties;

FIG. 17 is a view of a (female) brassier undergarment that will contain EMF/EMR/EHS barrier materials along with moisture blocking properties;

FIG. 18 is a view of a (female) bralette undergarment that will contain EMF/EMR/EHS barrier materials along with moisture blocking properties;

FIG. 19 is a view of a (female) body slip undergarment that will contain EMF/EMR/EHS barrier materials along with moisture blocking properties;

FIGS. 20 (A & B) is a view of a (unisex) infant onesie undergarment that will contain EMF/EMR/EHS barrier materials along with moisture blocking properties;

FIGS. 21 (A & B) is a view of a (unisex) thermal shirt undergarment that will contain EMF/EMR/EHS barrier materials along with moisture blocking properties;

FIGS. 22 (A & B) is a view of a (male) disposable absorption underwear that will contain EMF/EMR/EHS barrier materials along with moisture blocking properties;

FIGS. 23 (A & B) is a view of a (female) disposable absorption underwear that will contain EMF/EMR/EHS barrier materials along with moisture blocking properties;

FIG. 24 is a view of the genital inner panel frame of fabric for the undergarment embodiment that will be used in the application process for adult disposal garments that will contain EMF/EMR/EHS barrier materials along with moisture blocking properties;

FIG. 25 is a view of a genital inner panel frame for the undergarment embodiment that will be used in the application process for cloth diapers and/or training pants that will contain EMF/EMR/EHS barrier materials along with moisture blocking properties;

FIG. 26 is a view of a infant and toddler cloth absorption diaper that will contain EMF/EMR/EHS barrier materials along with moisture blocking properties; This embodiment may be conformed to gender specific embodiments and/or developed as a unisex pattern

FIGS. 27 (A & B) is a view of a toddler absorption training pant that will contain EMF/EMR/EHS barrier materials along with moisture blocking properties; This embodiment may be conformed to gender specific embodiments and/or developed as a unisex pattern

### DETAILED DESCRIPTION OF THE PREFERRED EMBODIMENTS

The following description includes many unambiguous details. The inclusion of such details are for the purpose of illustration only and should not be understood to limit the invention. Similarly, certain features which are well known in the art are not described in detail in order to avoid complication of the subject matter of the present invention. Additionally, it will be understood that features in one embodiment may be combined with features in other embodiments of the invention. Certain features may be interchanged with similar devices or features not mentioned yet which perform the same of similar functions. It is therefore intended that such modifications and variations are included within the totality of the present invention.

It should further be understood that the invention is not limited in its application to the details of construction and to the arrangements of the components set forth in the following description or illustrated in the drawings. The invention is capable of other embodiments and of being practiced and carried out in various ways. Furthermore, it should be understood that the terminology and phrases employed herein are for the purpose of description and should not be regarded as limiting. For example, as used herein, “properties, elements, and features” as well as, ‘blocked, blocking, barrier, management, and control,” may be used interchangeably throughout the description of the embodiments for the invention. Thus, it is important, therefore that the invention be regarded as including equivalent constructions to those described herein, insofar as they do not depart from the spirit and scope of the present invention.

In some embodiments, the present invention may encompass a dual and/or multiple layer barrier garment to block EMF/EMR/EHS emitting features, while also serving as a moisture protecting barrier, Additionally, the undergarment/intimate apparel will act as the first layer of clothing that touches the genital or upper body torso. Specifically, the present invention comprises multi-layers of undergarment applications, for example, tank tops, sports shapewear, bras, brassieres, bralettes, sports bras, strapless, pull over/slip on, nursing, corset, camisoles, breast shielding pads, classic briefs, high-cut briefs, control briefs, hipsters, bikinis, boyshorts, female boxers, tanga, briefs, and body slip, bodysuits wear, tanks, undershirts, t-shirts, long underwear, tube tops, for females, boxers, briefs, boxer briefs, trunks, jockstraps, bikinis, tank tops, undershirts, t-shirts, and long underwear, for males, as well as unisex thermal undershirt and regular undershirt apparel, adult disposal products for both genders, as well as infant onsies, diapers, and toddler training pants.

The embodiments in this invention will comprise varying composites of the fabrics that yield a EMF/EMR/EHS blocking element safe for direct human skin contact, as well as one or more of the microfiber or antimicrobial moisture reducing properties. This invention may involve a consortium with any of the following elements to further the consistency and quality of the property: cotton, nylon, hybrid yarn, copper, silver shielding, and bamboo fibers that are similar in nature and will not be considered mutually exclusive to the merits of this invention. Micromodal, antimicrobial, and/or mesh fabric with Polygiene® controlling elements will be used as moisture blocking properties for the embodiments in this invention. Any of the embodiments may also be tailored with appealing cosmetic effects such as satin, lace, nylon, spandex, adhesives, elastic encasing lycra, cotton, and other eco-friendly matters as found conductive to the production. The embodiments will be breathable, environmentally safe, comfortable, and provide blended elements of the invention to block emitting radiation from the body, while also yielding some moisture barrier protection from bacteria, human sweat and body odor. Additional fabrics to assist in the final production of the embodiments such as hooks, clasp, snaps, buttons, bands, trims, and enclosures could include nickel free, nylon coated, and silicone plastic, cooper nickel mesh, swan, eco-friendly adhesives, non-toxic cotton, organically grown cotton, spandex, and polymide. Intimate apparel will have fashionable and aesthetical features, including lace, satin, lycra, mesh, to add to the cosmetic appeal of the product.

Undergarments will act as insulators in a three-fold method by initially protecting the genital, breast cavity, and upper torso areas, and serve as a moisture guard, as well as a protective barrier from emitting electromagnetic and radiofrequency environmental and technological radiation. In addition, some undergarments will be more suitable to the consumer that are at greater exposure risk as a result of over/prolonged exposure and need EMF/EMR/EHS attenuation at a higher level that are still deemed safe for the human skin may be developed specifically for work-related exposure. Thus, EMF/EMR/EHS composite fabrics will be developed based on their attenuation properties and interchangeable use of one or more of the microfiber or antimicrobial moisture reducing properties will be included.

Infant diapers, toddler pants, and adult disposable pants will be consist of a top sheet (outer surface) with EMF/EMR/EHS barriers, and a back sheet (outer surface) with EMF/EMR/EHS barriers to include a bridge and wall element and therein between an moisture management inner layer that will include the crotch region. The structure of the infant, toddler, and adult disposable embodiments will include having a top sheet, a back sheet, which is connected in a superposed relation, and a moisture management layer that connects the crotch, with bridge and wall elements to act as interconnecting regions. Secondary moisture management blocking element may be used to control for leakage, spoils, incontinence, and accidents, which will be placed in the crotch area. Furthermore, side seams may be in different/varying views with adhesive, sewed, sonic scaled, autogeneous, or cohesive bonds, ultrasonic or pressure welds. Embodiments for the present disclosure may include stretch tabs, leg bands, side flaps, elastic, elastane, barrier, gasketing cuffs, or fasteners as varying ways secure the embodiment and reduce leakage in the disposable absorbent embodiment.

The material may be a woven or non-woven including film material, a non-woven may include natural, cotton, and synthetic fibers, the embodiment may be necked, meltdown, spunbond that includes laminate, carded layers, hydroentangled or otherwise developed. The disposables may also have fibers such as polyolefins/polypropylene, polyamides, polyester and/or polyethylene as well as biocomponent and then composed into nonwoven sheets. Hydrophobic material and surfaces may include cellulose, sodium polyacrylate, silicone polymers or fluorinated polymers. Several materials may be comprised to develop layers of laminated together. Top and back sheets may include high loft nonwoven, apertured film and apertured nonwoven. Disposable embodiments may include storage cores of cellulosic airfelt material, microfiber glues, and absorbent gelling materials. The various embodiments of the diaper, toddler pant, and adult disposable wear may be manufactured in different components to the methods disclosed herein according to the present disclosure.

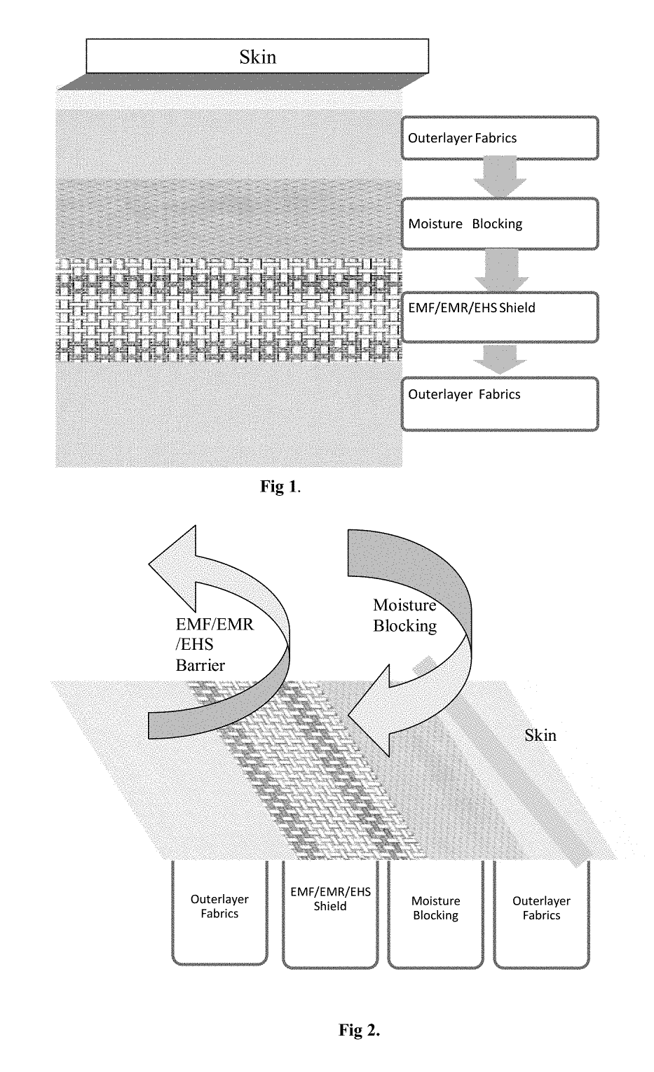

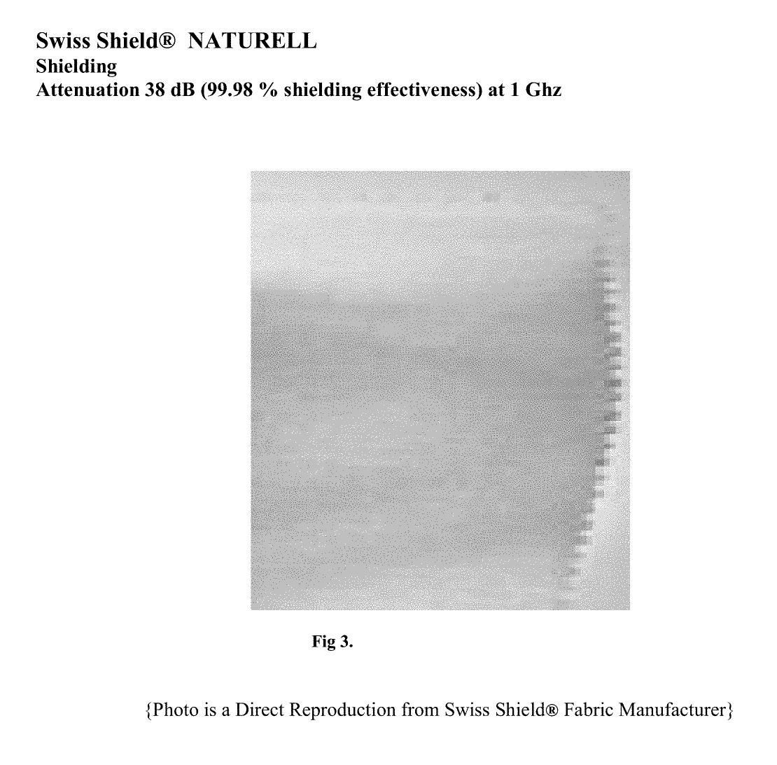

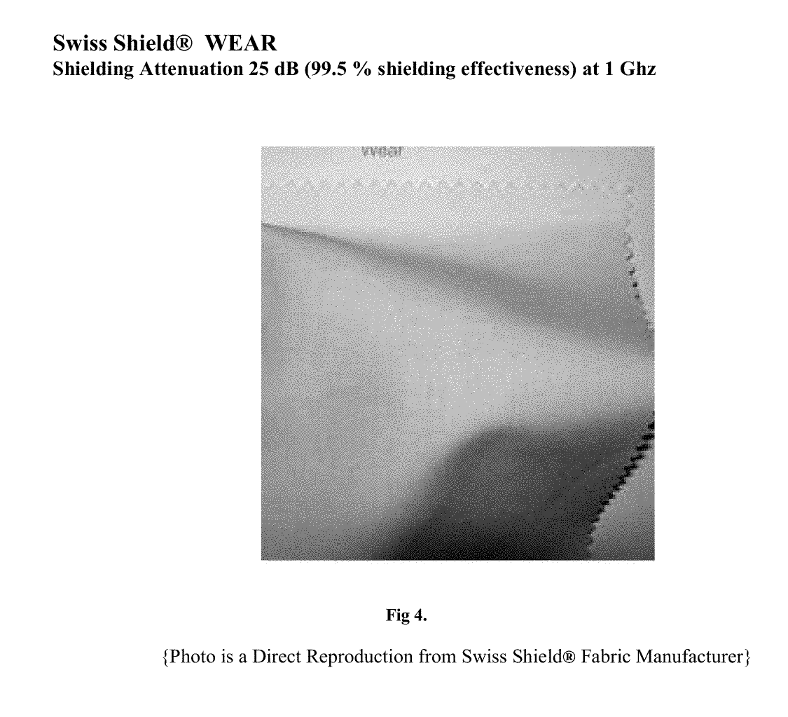

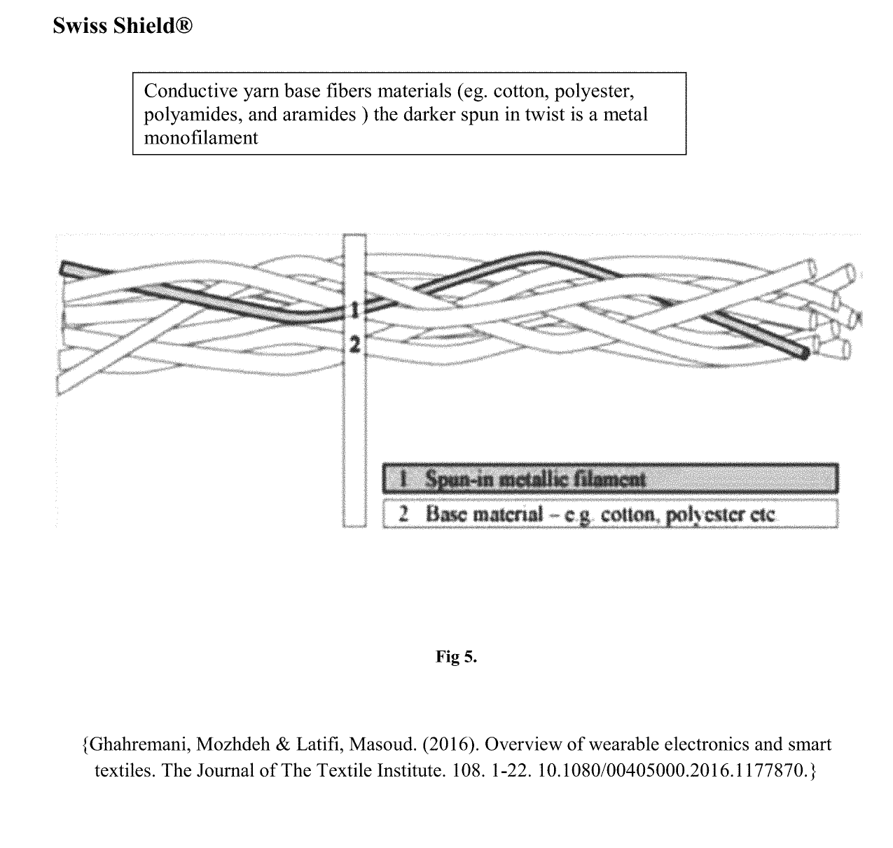

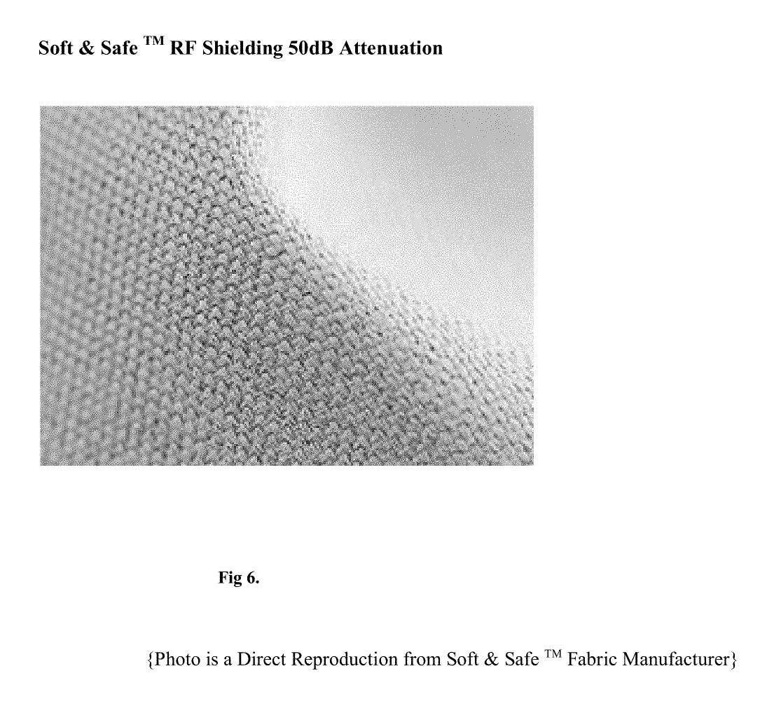

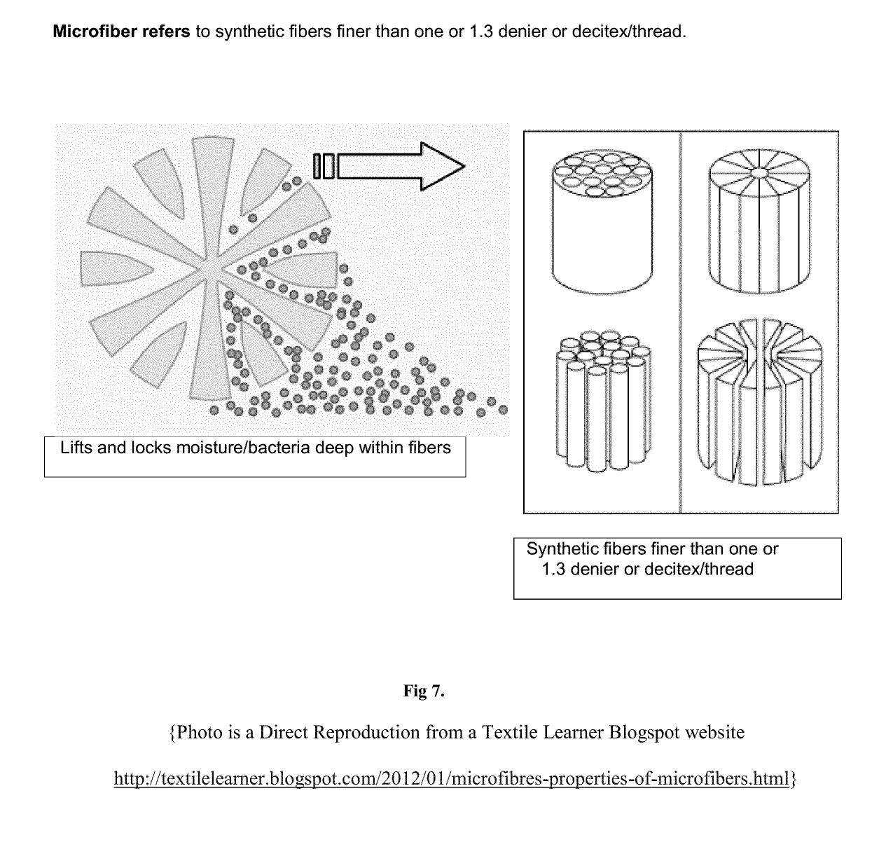

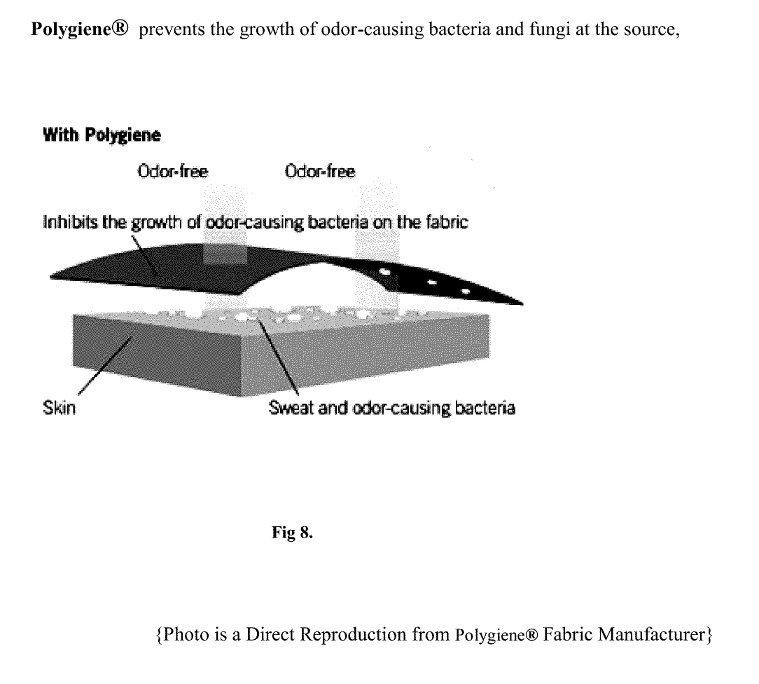

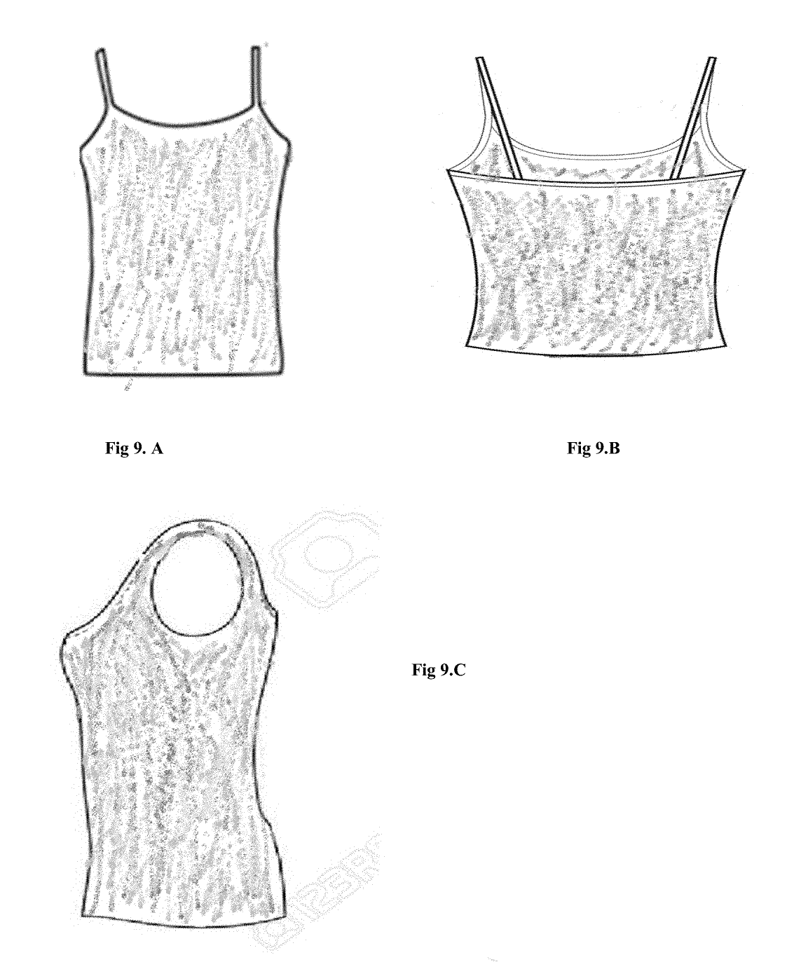

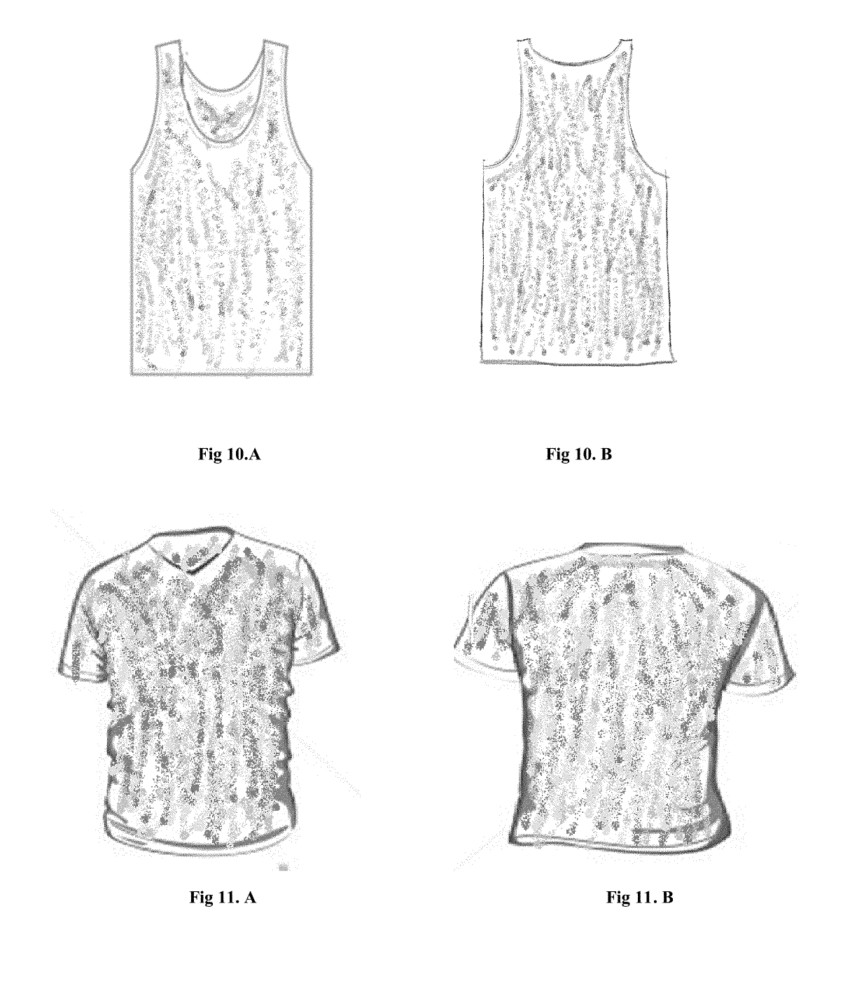

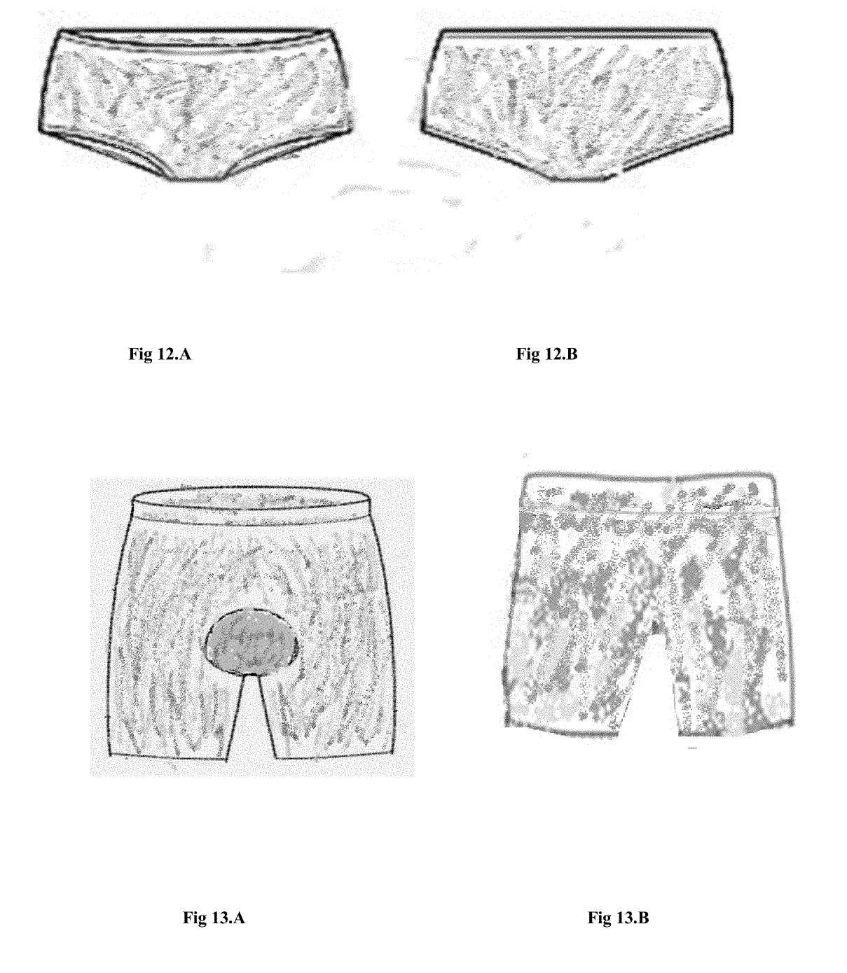

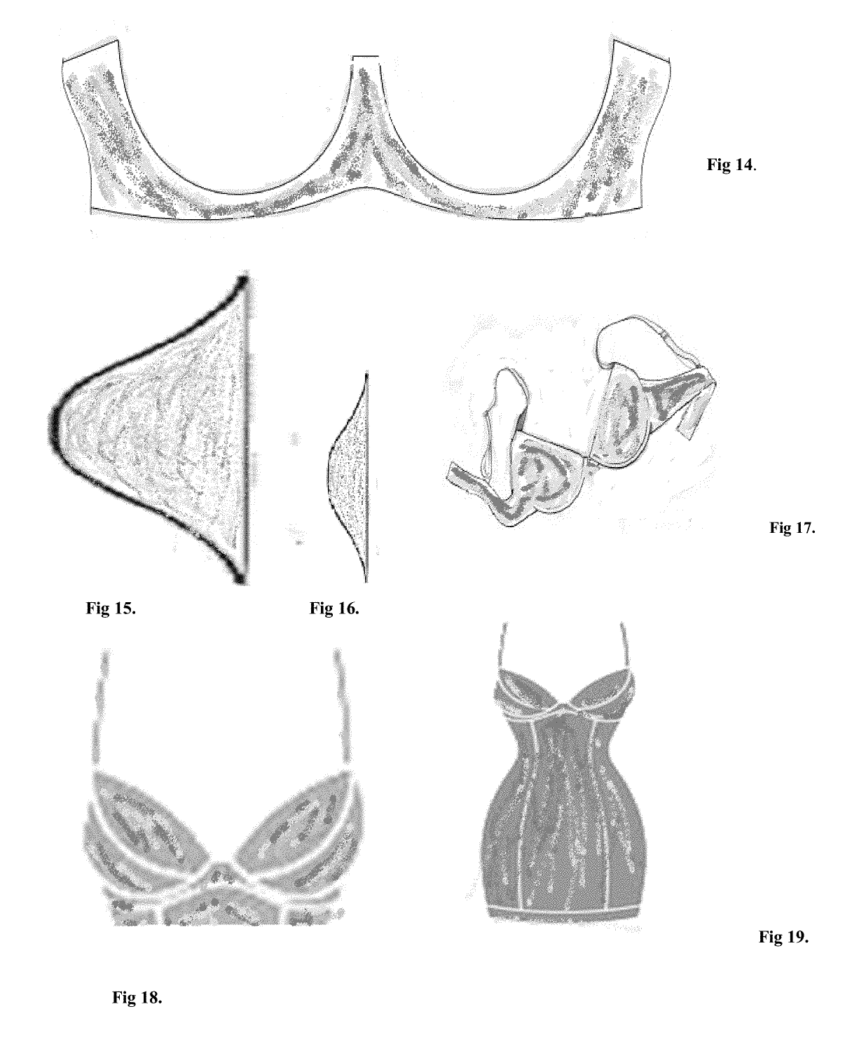

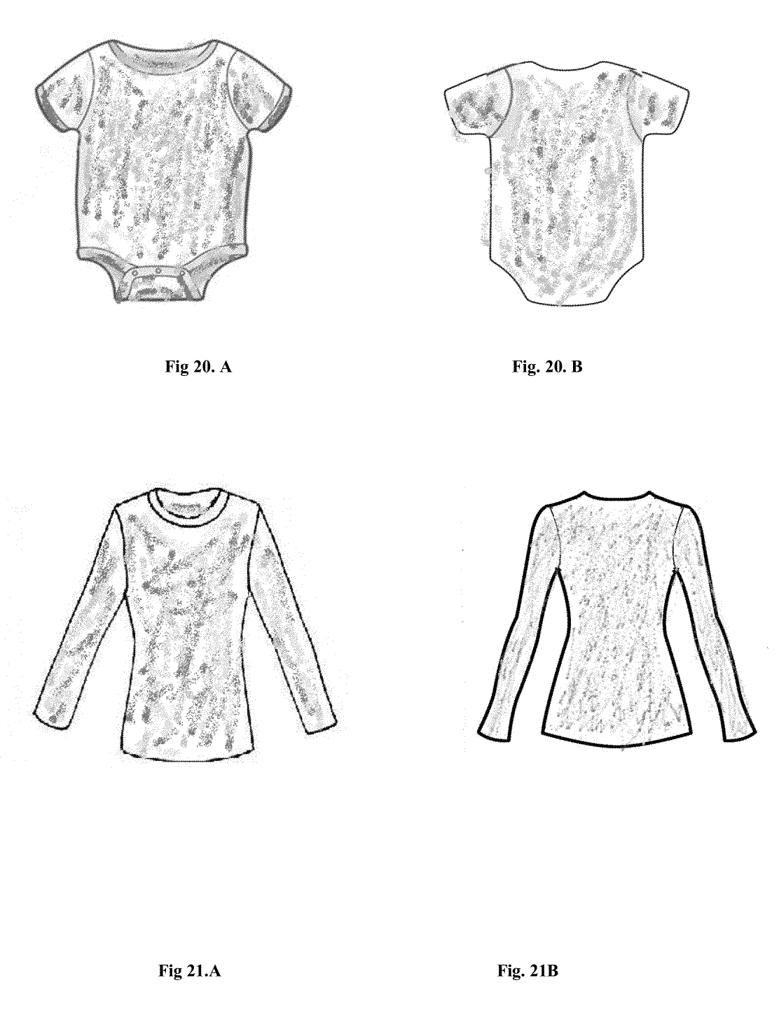

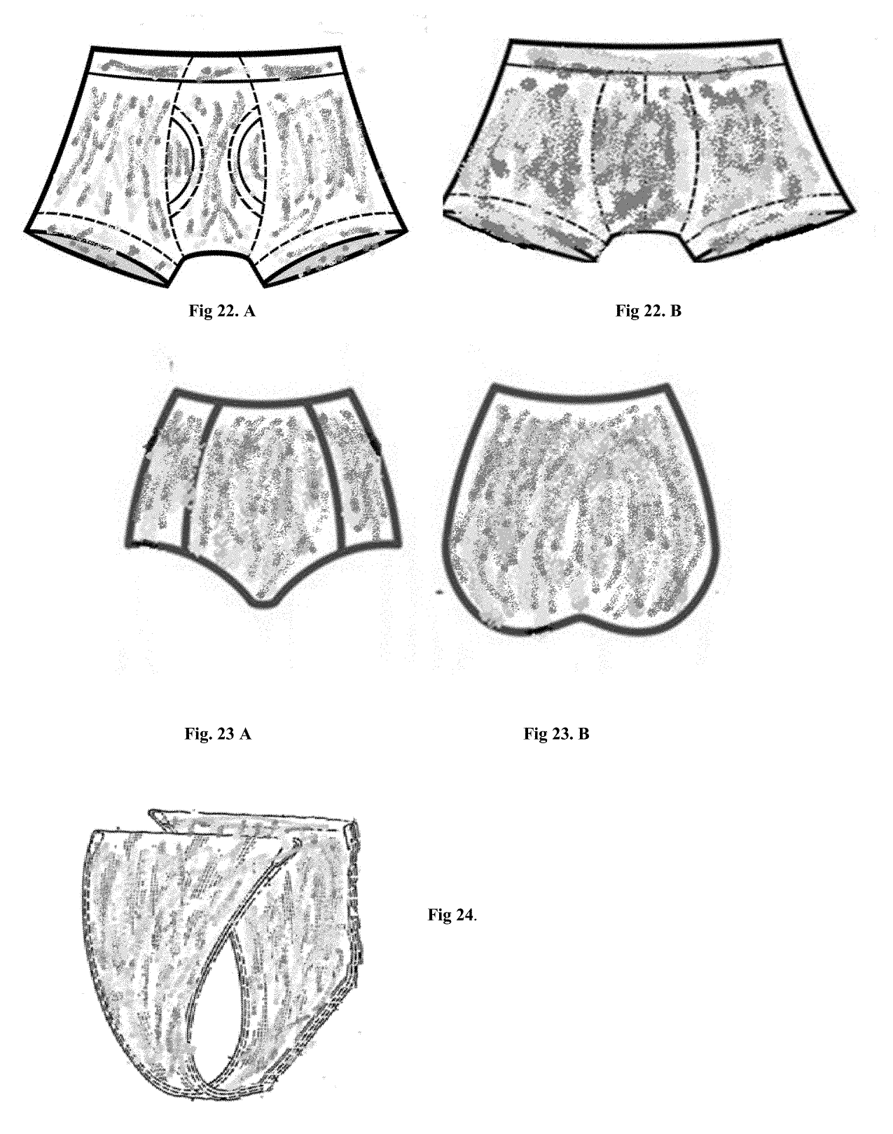

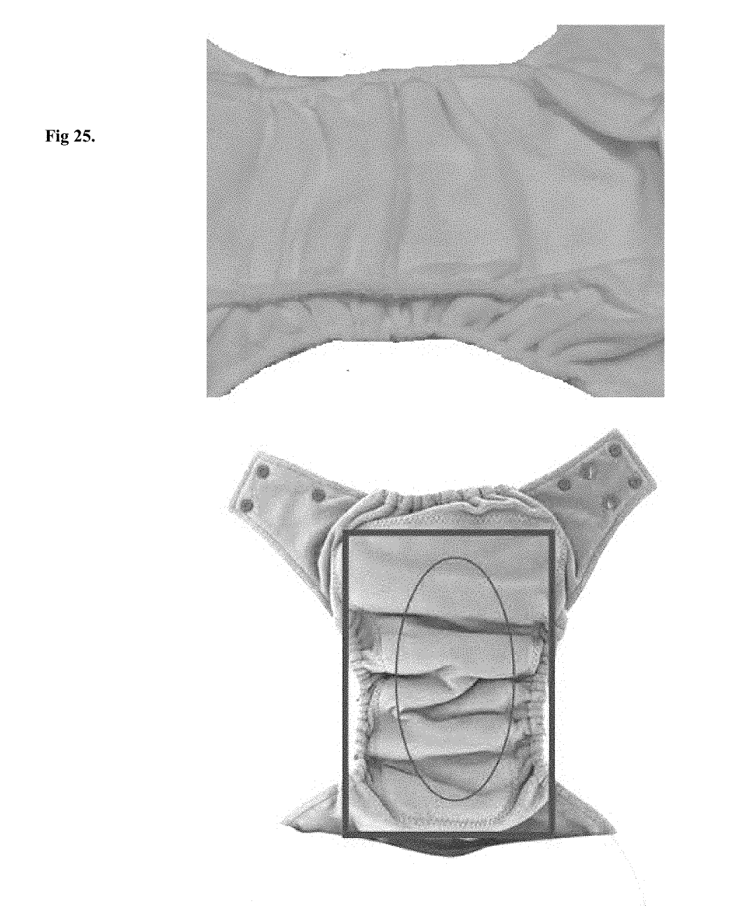

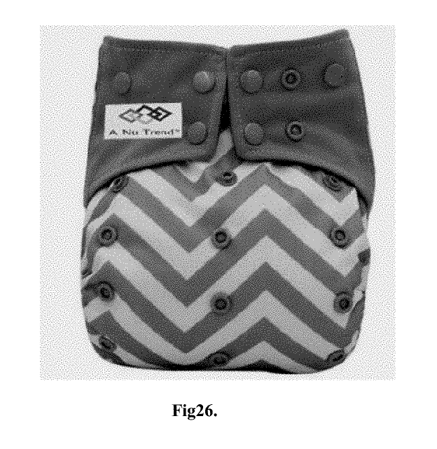

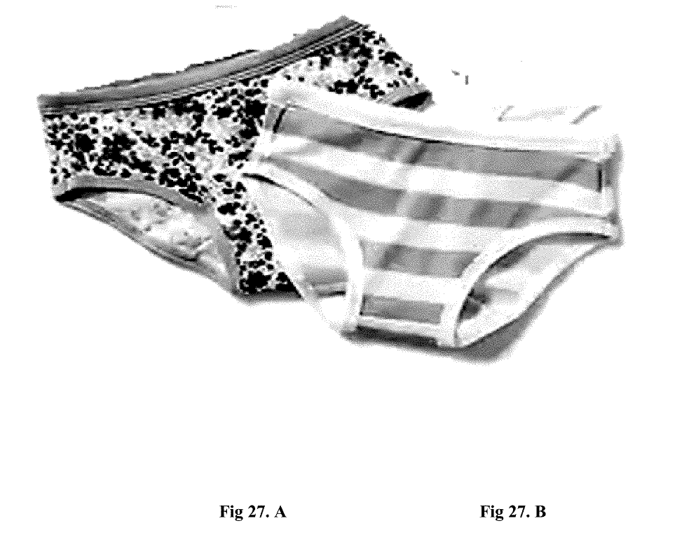

FIG. 1 illustrates a multi-layer view of the articles of fabric composition types that contain EMF/EMR/EHS radiation emitting blocked material coupled with microfiber or antimicrobial moisture properties intertwined between two or more articles of original fabric. This illustration displays the layout of how the multi-layer outline would depict an actual embodiment and show 1 being the human skin, 2 being an article of original fabric, such as polyester, nylon, cotton, spandex, lycra, polymide, organic cottons, or knits, 3 being a blocking moisture element, 4 being an EMF/EMR/EHS blocking fabric, and 2 being an article of original fabric, listed above in the same (no. 2) description.
FIG. 2 illustrates a dimensional view of the fabric composition types in order that contain EMF/EMR/EHS radiation emitting blocked material coupled with microfiber or antimicrobial moisture properties intertwined between two or more articles of original fabric. This illustration further displays the layout of how the multi-layer outline would depict an actual embodiment and show 1 being the human skin, 2 being an article of original fabric, such as polyester, nylon, cotton, spandex, lycra, polymide, organic cottons, or knits, 3 being a blocking moisture element, 4 being an EMF/EMR/EHS blocking fabric, and 2 being an article of original fabric listed above in the same (no. 2) description, with 5 showing the functional capability of the blocking moisture fabric illustrating the absorption properties, and 6 illustrating the retracting properties of the EMF/EMR/EHS fabric as these radicals are pushed outwardly away from the fabric and the body genital and torso areas.
FIG. 3 provides a written description of Swiss Shield® (Naturell) with 7 showing a 38 dB attenuation with 99.98% shielding effectiveness at 1 Ghz. and 8 illustrates the dB attenuation rates chart, and 9 is a chart demonstrating the dB and shielding performance for Naturell fabric.
FIG. 4 provides a written description of Swiss Shield® (Wear) with 10 showing a 25 dB attenuation with 99.5% shielding effectiveness at 1 Ghz that is capable of yielding a 29 dB depending on the mechanical outcomes from blended colors and/or dyes, 11 illustrates the dB attenuation rates chart, and 12 is a chart demonstrating the dB and shielding performance for Wear fabric.
FIG. 5 is a detail view of the Swiss Shield® 13 illustrated by the twist of a metal monofilament and 14 illustrates the conductive yarn base fibers (cotton, polyester, polyamides, and aramides, and 15 shows a brief description of the Swiss Shield® yarn conductive fibers.
FIG. 6 illustrates in 16 a description of the Soft & Safe™ shielding fabric with greater than 50 dB attenuation and 17 illustrates a preview of the fabric material, and 18 is a chart to report the dB and shielding performance for the Soft & Safe™
FIG. 7 illustrates in 19 a description of Microfibers of the synthetic fibers that are finer than one or 1.3 denier or decitex/thread as well as a listing of the characteristics of the microfibers. The illustration of 20 represents the synthetic fiber with moisture/bacteria being locked into the inner panels of the fibers. 21 shows an illustration of particles of moisture/bacteria, 22 is an illustration of synthetic fibers encased, 23 is another mode of synthetic fibers in a pinwheel design, 24 is an illustration of the fibers as they begin the absorption process, 25 is an illustration of the synthetic fibers without the casing.
FIG. 8 illustrates in 26 the benefits of the Polygiene® antimicrobial properties, and 27 is a portion of the illustration that supports the layering elements and skin as the 1st portion of the embodiment, and 28 shows the inner portion of the embodiment and demonstrates how perspiration, body odor, and bacteria, are produced from the skin. The illustration in 29 shows the Polygiene® antimicrobial properties that inhibit growth of odor causing bacteria on the fabric.
FIGS. 9 (A, B, & C) shows varying views 30 front, back and side of a (female) tank with both EMF/EMR/EHS blocked emitting applications and moisture barriers. The internal viewing (female) tank 31 shows the inward application of both EMF/EMR/EHS blocked emitting applications and moisture barriers. Illustrations in 32 show the trims, ridges, extensions, and enclosures. And 33 shows a side profile with shape contouring effects with both EMF/EMR/EHS blocked emitting applications and moisture barriers.
FIGS. 10 (A & B) shows the front and back views 34 of a (male) tank with both EMF/EMR/EHS blocked emitting applications and moisture barriers. Illustrations in 35 show the trims, ridges, extensions, and enclosures. And 36 is an illustration of the internal views of the (male) tank that supports the inward application of both EMF/EMR/EHS blocked emitting applications and moisture barriers.
FIGS. 11(A & B) provides an illustration 37 of front and back views of a (unisex) top with both EMF/EMR/EHS blocked emitting applications and moisture barriers. And 38 is an illustration of the internal views of the (unisex) top with both EMF/EMR/EHS blocked emitting applications and moisture barriers. Illustrations in 39 show the trims, ridges, extensions, and enclosures.
FIGS. 12 (A & B) provides an illustration 40 of front and back views of a (female) panty with both EMF/EMR/EHS blocked emitting applications and moisture barriers. Illustrations in 41 show the trims, ridges, extensions, waistband, and enclosures. And 42 is an illustration of the internal views of the (female) panty with both EMF/EMR/EHS blocked emitting applications and moisture barriers.
FIGS. 13 (A & B) provides an illustration 43 of front and back views of a (male) brief with both EMF/EMR/EHS blocked emitting applications and moisture barriers. And 44 is an illustration of the internal views of the (male) brief with both EMF/EMR/EHS blocked emitting applications and moisture barriers. Illustrations in 45 show the trims, ridges, extensions, waistband, and enclosures. The illustration in 46 shows the crotch liner/panel of the male (brief) with both EMF/EMR/EHS blocked emitting applications and moisture barriers.
FIG. 14 provides an illustration 47 under bridge and side panel base for a bra with both EMF/EMR/EHS blocked emitting applications and moisture barriers. And 48 is an illustration of the trims, ridges, extensions, and enclosures. The illustration in 49 shows the inner folds, shaping, and under bridge with side panel base of the bra insert with both EMF/EMR/EHS blocked emitting applications and moisture barriers.
FIG. 15 provides an illustration 50 of a breast cupping. molded, contour form with both EMF/EMR/EHS blocked emitting applications and moisture barriers. Illustrations in 51 show the trims, ridges, extensions, and enclosures.
FIG. 16 provides an illustration 52 of a nipple shield pad that will contain both EMF/EMR/EHS blocked emitting applications and moisture barriers, as well as eco-friendly adhesives. Illustrations in 53 show the trims, ridges, contouring effects, and enclosures.
FIG. 17 provides an illustration 54 of a (female) brassier with both EMF/EMR/EHS blocked emitting applications and moisture barriers. In 55 the views show the under bridge and side panel base for the brassier with both EMF/EMR/EHS blocked emitting applications and moisture barriers. And 56 is an illustration of the trims, ridges, extensions, and enclosures. 57 shows a viewing of the brassiere's straps and 58 is an illustration of the brassiere clasp side panel with both EMF/EMR/EHS blocked emitting applications and moisture barriers.
FIG. 18 provides an illustration 59 of a (female) bralette with both EMF/EMR/EHS blocked emitting applications and moisture barriers. And 60 is the under bridge base for the bralette with both EMF/EMR/EHS blocked emitting applications and moisture barriers. Illustrations in 61 shows the trims, ridges, contouring effects, and enclosures. 62 shows a viewing of the bralette's straps and 63 is an illustration of the bralette contouring side panel with both EMF/EMR/EHS blocked emitting applications and moisture barriers.
FIG. 19 provides an illustration 64 of a (female) body slip with both EMF/EMR/EHS blocked emitting applications and moisture barriers. The views in 65 illustrate the body contouring and shapewear with both EMF/EMR/EHS blocked emitting applications and moisture barriers. Illustrations in 66 shows the trims, ridges, extensions, and enclosures. The views in 67 show the body slip straps.
FIGS. 20 (A & B) provides an illustrations 68 of front and back views of a (unisex) infant onesie with both EMF/EMR/EHS blocked emitting applications and moisture barriers. And 69 is an internal view of the inner portion of the embodiment with both EMF/EMR/EHS blocked emitting applications and moisture barriers. Illustrations in 70 shows the trims, ridges, extensions, and enclosures.
FIGS. 21 (A & B) provides an illustration 71 of front and back views of a (unisex) thermal shirt with both EMF/EMR/EHS blocked emitting applications and moisture barriers. And 72 is an internal view of the inner portion of the embodiment with both EMF/EMR/EHS blocked emitting applications and moisture barriers. Illustrations in 73 shows the trims, ridges, extensions, and enclosures.
FIGS. 22 (A & B) provides an illustration 74 of front and back views of a (adult male) disposable absorption pant with both EMF/EMR/EHS blocked emitting applications and moisture barriers. And 75 is an internal view of the inner portion of the embodiment with both EMF/EMR/EHS blocked emitting applications and moisture barriers. Illustrations in 76 is the crotch liner/panel of the disposable absorption pant with both EMF/EMR/EHS blocked emitting applications and moisture barriers. Illustrations in 77 shows the trims, ridges, extensions, waistbands, and enclosures.
FIGS. 23 (A & B) provides an illustration 78 of front and back views of a (adult female) disposable absorption pant with both EMF/EMR/EHS blocked emitting applications and moisture barriers. And 79 is an internal view of the inner portion of the embodiment with both EMF/EMR/EHS blocked emitting applications and moisture barriers. Illustrations in 80 shows the trims, ridges, extensions, waistbands, and enclosures.
FIG. 24 provides an illustration 81 of absorption properties of a inner lining panel that may be used as a portion of an embodiment for a disposable portion in the present disclosure and will include both EMF/EMR/EHS blocked emitting applications and moisture barriers. Illustrations in 82 shows the trims, ridges, extensions, and enclosures. And 83 is an internal view of the inner portion of the embodiment with both EMF/EMR/EHS blocked emitting applications and moisture barriers.
FIG. 25 provides an illustration 84 of a infant and toddler absorption of an inner lining panel for use in infant and toddler diapers and for toddler pants with both EMF/EMR/EHS blocked emitting applications and moisture barriers. And 85 is an internal view of the inner portion of the embodiment with both EMF/EMR/EHS blocked emitting applications and moisture barriers. Illustrations in 86 shows the trims, ridges, extensions, and enclosures.
FIG. 26 provides an illustration 87 of a infant and toddler absorption diaper with front and side views that will include both EMF/EMR/EHS blocked emitting applications and moisture barriers. This embodiment may be conformed to gender specific embodiments and/or developed as a unisex pattern. And 88 is an internal view of the inner portion of the embodiment with both EMF/EMR/EHS blocked emitting applications and moisture barriers. Illustrations in 89 shows the trims, ridges, extensions, waistbands, and enclosures. And in 90 is a view of the clasp/clamp/latch elements for the embodiment of the present disclosure. Illustrations in 91 shows the adhesive/sealants for the embodiment of the present disclosure.
FIGS. 27(A & B) provides an illustration 92 of a toddler absorption pant with front and back views that will include both EMF/EMR/EHS blocked emitting applications and moisture barriers. This embodiment may be conformed to gender specific embodiments and/or developed as a unisex pattern. Illustrations in 93 show the crotch/liner panel with both EMF/EMR/EHS blocked emitting applications and moisture barriers. Illustrations in 94 show the trims, ridges, extensions, waistbands, and enclosures. And in 95 is an internal view of the inner portion of the embodiment with both EMF/EMR/EHS blocked emitting applications and moisture barriers.

The goal of this invention is to act as a barrier for emitting electromagnetic and radio frequency properties as well as keep the intimate body regions dry and cool with antimicrobial blocking fabric elements. Inner linings will include front and back panel sides and under lining sections of the product with the essential aspects of EMF/EMR/EHS blocked emitting applications and moisture barriers. Protective barriers will be encased in the front, back, and side panels, as well as within the crotch area within the embodiments of this invention.

The claims to support this patent will show novelty and non-obviousness based on the following (6) facts:
1). The novelty for the claims in this patent will not be faltered, because several of the aversive risk factors from the growth of wireless and cellular devices were unknown and the contributory environmental and public health issues are just now becoming paramount. 2). In addition, the community, schools, parents, advocacy groups and panels have made the health and environment issues relevant in the past five years with the ongoing reports from consumers, medical and health care providers, and ecological investigators. 3). Furthermore, the claims in this patent will reduce the daily exposure for consumers, as well as benefit the health care sector and the environment by offering products that lessen the EMF/EMR exposure. Ultimately, this will lower the insurmountable health care costs as well as offer products that are eco-friendly with low risk to the existing pollutions and toxins in the ecological system. 4). In addition, the revolution of smart textiles have become increasingly popular based on their distinct technological advances and functionality. This patent is specifically tailored to address and protect the wearer from environmental hazards from pulsating radiofrequency waves with the use of these smart textile compositions. 5). Furthermore, consumers are attracted to aesthetical designed undergarments that have eco-friendly and moisture-resistant properties. Prior art lacked any aesthetical appeal, was developed for only static effects, failed to consider the potential human skin irritation based on high power density textiles, or was designed based on silver fabric particles that would later draw biohazard and ecological concerns. 6). Lastly, the commercial success of this patent will display the market's interest in having undergarments that can offer the EMF/EMR/EHS benefits as described, and further support its non-obviousness.

The present invention has been illustrated and described, it would be apparent to those skilled in the art that various other changes and modifications can be made without departing from the spirit and scope of the invention. It is therefore intended to cover the appended claims all such changes and modifications that are within the scope of this invention.

What is claimed is:

1. An undergarment with multi-layer barriers providing attenuation that will range from 20 dB up to 50 dB consisting of any of the following (Swiss Shield®—Naturell or Wear and/or Soft & Safe™) composite materials or similar shielding textile composites and a moisture management element that includes microfiber synthetic fibers finer than 1.3 denier or decitex/thread and/or with Polygiene®—antimicrobial blocking applications to protect the upper or lower torso of the body cavity. More notably, the proclamation of the above (6) facts will be incorporated and supported within this claim.

2. It is claimed that Swiss Shield®, EMF/EMR/EHS is certified by Okeo-Tex-100 (i.e. meeting the standards of being gentle enough for baby underwear) and the following composite materials Naturell and Wear will be used in some of the embodiments of this invention.

3. It is claimed that Soft & Safe™ will be used in the composition for production in embodiments where consumers require additional protection from prolonged or over exposure, thus decreasing the susceptibility and high sensitivity from adverse health effects due to persistent EMF/EMR exposure.

4. It is further claimed that consumers will have textile shielding selection options in particular for safety risk, type of exposure, as well as length or over exposure, and will be informed of the specification of the attenuating effects of each embodiment of this invention.

5. It is claimed that all EMF/EMR/EHS blocking and/or shielding fabrics will be safe based on textile certification for the human skin and the manufacturing of the embodiments described for this invention

6. It is claimed that some composite fibers for EMF/EMR/EHS may be altered with safe eco-friendly organic, biodegradable and cotton blend dyes for cosmetic appeal that will reduce ecological harm.

7. It is claimed that embodiments will included composite fabrics that will help to channel moisture away from the surface of the skin and thus provide management and/or control of the antibacterial properties as well as may provide odor blocking elements that include either microfiber, Polygiene®, or other antimicrobial features.

8. It is claimed that the undergarment will have liner frames comprising at least one layer of a EMF/EMR/EHS shielding barrier materials along with moisture blocking properties of claim 1;
each material configured to conform with at least part of the portion of the garment overlaying the torso area, one surface of said layer facing toward an inside portion of at least a part of said garment, and one surface of said layer facing toward at least a part of the torso area of claim 1;
at least part of the said liner comprising EMF EMF/EMR/EHS barrier materials along with moisture blocking properties of claim 1;
at least part of the said liner will protect at least part of the torso area emitting EMF/EMR/EHS barrier materials along with moisture blocking properties of claim 1;
at least part of the said liner may be composed of disposable properties for the lower torso area emitting EMF/EMR/EHS barrier materials along with moisture blocking properties of claim 1.

9. The attenuating and moisture management articles in claim 1, comprising of a (female) (male) or (unisex) tank undergarment.

10. The attenuating and moisture management articles in claim 1, comprising of a (unisex) or (gender specific) shirt that may be short, long, and/or thermal to function as an undergarment.

11. The attenuating and moisture management articles in claim 1, comprising of a (female) or (unisex) panty undergarment to cover the lower torso.

12. The attenuating and moisture management articles in claim 1, comprising of a (male) or (unisex) brief undergarment to cover the lower torso.

13. The attenuating and moisture management articles in claim 1, comprising of a (female) under bridge and siding panel base for a bra forming undergarment that may consist of breast cupping, molded, contour form, nipple shield to serve in the brassier or bralette region.

14. The attenuating and moisture management articles in claim 1, comprising of a (female) (unisex) or infant body covering or slip to function as an undergarment.

15. The attenuating and moisture management articles in claim 1, comprising of a (male) or (female) disposable and/or absorption underwear.

16. The attenuating and moisture management articles in claim 1, comprising of a infant and toddler disposable and/or absorption diaper, conformity to gender specific embodiments and/or developed as a unisex pattern will be optional.

17. The attenuating and moisture management articles in claim 1, comprising of a toddler disposable and/or absorption training pant, conformity to gender specific embodiments and/or developed as a unisex pattern will be optional.
